SEQUENCE LISTING (10 sequences)

SEQ ID NO: 1 - DNA, 2846 bp, Litchi sinensis
  misc_feature (2670)..(2670): n is a, c, g, or t
ggagtgcaggattagaaaatttggaagtagtgagcgatgtcggagaagatgaaaatggcggtggtcggtggcggaatgagtggagtggtggcagctcatgtacttgcaaaagcaggtgttgaggttgtagtgtatgagaagaagaactacctgggtggccaagctaagattgttacctttgatggcgtcgatatagaccttggatgcatgttctttaacggtatgacgtgttcgaatatgatggagatgtctgagagtttagggcttgaaatggagatagtagagatgtcattctcagtgagcttagaccaaggccaaggctgtgaatggggtaccagaaacgggttgactagtttgtttgcacagaagaagaatgtgtttaatccttacttttggcaaatgcttcgagaaataactaagtttaagaaggatgccatcagttattttgaaatactcgaaaacaacccagacatcaattatgatgaaaccttggagcaatttgtgaagtcacataattactctgaattatttcagaaggcttatcttattccagtatgttgtataatatggtcttgccccgcggagcgagttatgagctttcctattctctatgttttttcagtttatcgcaatcatcgtctatttcagctccttggtggcccacaatggctaactactagatggcgttcagataactattacaaaagggtcaaggaacaacttgagaactggggctgtcaaataagaactagtagtgaggtgtattctgtttcgccagttgatgaaggttgtgcagtaatatgtgacgatggttcaaaagaattttacaatggatgcataatggccgttcgtgccccggatgctttgaaaatgctaggaagccaagcaacatgggatgaattaaggatactaggtgctttccagtatgtctgcagtgatatttttcttcaccgtgataagaattttatgccccgaaatcaagcagcatggagcgcatgcaactttatgcgaatcacaggcagcaaaactgtatgcttgacatactggctcaatgtacttcaggatatcggtgaaaagagttcaccttttcttgtaacgctcaatcctgaacatacaccagaacagacgttactgaagtggtctactggccatcctgtcccatctgttgctgcatcaaaagcttcacgtgagcttgatcttattcaggggaaaagaggaatttggttttgtggagcttatcaaggttatggcttccatgaggatcaactaaagtcgggattgattgctgcacatagtttgctgggaaaaagttgtgctattttgagcaaaccaagacacatggcaccttccatgatggaaacgggggcacggctttttgttactagatttcttggacaatatatcattgctggccgtttgatattgatggaagatggaggcgcagtttttaaatttgagggaacgaggaaaaattgttgtctagaaactatattgagaattcataatcctgaattttattggaaggttatgactgaagcagatttaggctttgcagattcatatatcaatggagatttttcttttgtggataaagataagggtctcctaaatctttttctgattttcatcgccaatagccatttggaccctttacgtgcaaaattgaaccagaaaaggggttggtggtcaccgatgttcttcactgctggtattgcatctgcaaaatatttctttcgtcatattttaaaccgaaatactcttacacaagctcgtaggaatatctctcgccactatgatctgagcaatgatctttttgctctcttcttggacgaatcaatgacatactcatgtgcagtgttcaagagtgaagatgaagatttgaaagttgcacaaatgagaaaactatcccttctgattgagaaagcaagaattagtaaggagcacgaagttcttgacatcggttgtgggtggggatccttagctatagaagccgtcaaacgaactggatgcaaatacactggcattagtctgtccgtggagcaaattaaatatgcagatatcaaagtgaaggaagccggccttcaggaccaagtaacattaattcactgtgactaccgccaactacctaaaaccaacaaatacgacagaattatttccgtcgggatgatagactccgttggccatgaatacttcgaggagttctttggttgttgtgaatcactactggcagaagatggcctttttgttctacagtatatatcagtaccggatcagcactataatgaacataggctgagtccaggttttataaaggaatacatttttcctggtggatgcttgccttcattgaataggataacaacagctatgacttctgcctccagactctgtgtggagcatgtggaaaatattggaatccattaccaccaaacactgagatggtggagaaaaaatttgttggagagacagagcgaagtccttgctttgggatttgatgaaaagttcattcgaacatgggaatattattttgattattgtgctgctggattcaagtctcgtacattgggagattatcagatcgtattttcacgtcctggcaatgttgctgcattcactaatccatacaaaggctttccttcaactgattctgttatgtaaagagagcggagngcatatctnctctccctatgttgcctcaattggtgatcagtttgatgatgaataaatgtgattaaccaataaaaggccancaacttaaaaggaatctgaagtagatatgtattgatcatcttcagaaattcttaccaataagtgatgcnctattattccaaaaaaaaaaaaaaaaa

SEQ ID NO: 2 - protein, 873 aa, Litchi sinensis
MSEKMKMAVVGGGMSGVVAAHVLAKAGVEVVVYEKKNYLGGQAKIVTFDGVDIDLGCMFFNGMTCSNMMEMSESLGLEMEIVEMSFSVSLDQGQGCEWGTRNGLTSLFAQKKNVFNPYFWQMLREITKFKKDAISYFEILENNPDINYDETLEQFVKSHNYSELFQKAYLIPVCCIIWSCPAERVMSFPILYVFSVYRNHRLFQLLGGPQWLTTRWRSDNYYKRVKEQLENWGCQIRTSSEVYSVSPVDEGCAVICDDGSKEFYNGCIMAVRAPDALKMLGSQATWDELRILGAFQYVCSDIFLHRDKNFMPRNQAAWSACNFMRITGSKTVCLTYWLNVLQDIGEKSSPFLVTLNPEHTPEQTLLKWSTGHPVPSVAASKASRELDLIQGKRGIWFCGAYQGYGFHEDQLKSGLIAAHSLLGKSCAILSKPRHMAPSMMETGARLFVTRFLGQYIIAGRLILMEDGGAVFKFEGTRKNCCLETILRIHNPEFYWKVMTEADLGFADSYINGDFSFVDKDKGLLNLFLIFIANSHLDPLRAKLNQKRGWWSPMFFTAGIASAKYFFRHILNRNTLTQARRNISRHYDLSNDLFALFLDESMTYSCAVFKSEDEDLKVAQMRKLSLLIEKARISKEHEVLDIGCGWGSLAIEAVKRTGCKYTGISLSVEQIKYADIKVKEAGLQDQVTLIHCDYRQLPKTNKYDRIISVGMIDSVGHEYFEEFFGCCESLLAEDGLFVLQYISVPDQHYNEHRLSPGFIKEYIFPGGCLPSLNRITTAMTSASRLCVEHVENIGIHYHQTLRWWRKNLLERQSEVLALGFDEKFIRTWEYYFDYCAAGFKSRTLGDYQIVFSRPGNVAAFTNPYKGFPSTDSVM

SEQ ID NO: 3 - DNA, 1071 bp, Litchi chinensis
atggaaacgataatgcaggcaccctacgatgcgacagtgcggctgatgctagcttcactggagaggaatctgctgccggacgttgttatacggagactgactcgcctgctattggccagtcgccttcgctcctgttacgaaccttcttctcaactccaactcgcccacctcctacacttcgtacattctcttaaggagatgcctatagctatcgagacggacaagcccaaatcacagcattatgaactgccaatctccttcttcaagctggtgcttggacagaatcttaaatacagctgctgttacttctctgataagtcaagcaccttagaggatgctgagaaagaaatgctggagctgtactgtgagaggtgtcagttaaaagatggccacactgttcttgatgttggatgtggttgggggtcactttctctatacattgcccaaaagtatagtaactgcaagattacagggatatgcaattcaagaactcaaaaagcatacattgaggagcaatgtcgggttcttgagattcagaatgttgagatcattcaagcagatattagcacatttgaaatggaggcatcctacgaccgaatattctccattgaaatgtttgagcatatgaagaattaccaagatcttcttaagaagatatccagctggatgaaacaggatagccttctctttgttcaccatttttgccacaaaacatttgcctatcactatgaggtcacgaatgaagaagactggattactaagtacttcttcagtgggggtacgatgccttcagctaatctgcttctttatttccaggatgatgtttctgttgtcgatcactggcttgtcaatgggaagcattatgcacaaacaagtgaagagtggctcaaaagaatggaccagaacatggcttccataaagccaataatggagtcaacttatggcaaagatcaggctgtcaagtggactgtttattggagaacattcttcattgctgttgcagaacttttcggatacagtgaaggagaagaatggatggtcactcatttcttattcaaaaagaagaactga

SEQ ID NO: 4 - protein, 356 aa, Litchi sinensis
METIMQAPYDATVRLMLASLERNLLPDVVIRRLTRLLLASRLRSCYEPSSQLQLAHLLHFVHSLKEMPIAIETDKPKSQHYELPISFFKLVLGQNLKYSCCYFSDKSSTLEDAEKEMLELYCERCQLKDGHTVLDVGCGWGSLSLYIAQKYSNCKITGICNSRTQKAYIEEQCRVLEIQNVEIIQADISTFEMEASYDRIFSIEMFEHMKNYQDLLKKISSWMKQDSLLFVHHFCHKTFAYHYEVTNEEDWITKYFFSGGTMPSANLLLYFQDDVSVVDHWLVNGKHYAQTSEEWLKRMDQNMASIKPIMESTYGKDQAVKWTVYWRTFFIAVAELFGYSEGEEWMVTHFLFKKKN

SEQ ID NO: 5 - protein, 459 aa, Litchi sinensis
MIAAHSLLGKSCAILSKPRHMAPSMMETGARLFVTRFLGQYIIAGRLILMEDGGAVFKFEGTRKNCCLETILRIHNPEFYWKVMTEADLGFADSYINGDFSFVDKDKGLLNLFLIFIANSHLDPLRAKLNQKRGWWSPMFFTAGIASAKYFFRHILNRNTLTQARRNISRHYDLSNDLFALFLDESMTYSCAVFKSEDEDLKVAQMRKLSLLIEKARISKEHEVLDIGCGWGSLAIEAVKRTGCKYTGISLSVEQIKYADIKVKEAGLQDQVTLIHCDYRQLPKTNKYDRIISVGMIDSVGHEYFEEFFGCCESLLAEDGLFVLQYISVPDQHYNEHRLSPGFIKEYIFPGGCLPSLNRITTAMTSASRLCVEHVENIGIHYHQTLRWWRKNLLERQSEVLALGFDEKFIRTWEYYFDYCAAGFKSRTLGDYQIVFSRPGNVAAFTNPYKGFPSTDSVM

SEQ ID NO: 6 - DNA, 23 bp, Artificial
  Primer P18-P4
aaactgcgcctccatcttccatc

SEQ ID NO: 7 - DNA, 27 bp, artificial
  Primer P18-P1
tcatgattgctgcacatagtttgctgg

SEQ ID NO: 8 - DNA, 22 bp, artificial
  Primer LcfaTrev
agatgcaataccagcagtgaag

SEQ ID NO: 9 - DNA, 25 bp, artificial
  primer RESrev
cgagtgacacttgatgtgaacatgc

SEQ ID NO: 10 - DNA, 20 bp, artificial
  primer RESfor
ggtcaggttgcctaggaagc

(19) United States
(12) Patent Application Publication
(10) Pub. No.: US 2008/0155714 A1
(43) Pub. Date: Jun. 26, 2008

(54) Plant Cyclopropane Fatty Acid Synthase Genes and Uses Thereof

(75) Inventors: Eric Gontier, Germiny (FR); Brigitte Thomasset, Compiegne (FR); Emma Wallington, Cambridge (GB); Jeroen Wilmer, Levignac (FR)

Correspondence Address: CONNOLLY BOVE LODGE & HUTZ, LLP, P O BOX 2207, WILMINGTON, DE, 19899, US

(73) Assignees: Total France, Puteaux (FR); Limagrain Agro Industrie, Chappes (FR)

(21) Appl. No.: 11/884,605

(22) PCT Filed: Feb. 16, 2006
(86) PCT No.: PCT/EP2006/060030

§ 371 Date: Oct. 15, 2007

(30) Foreign Application Priority Data
Feb. 18, 2005 (EP) 05101271.4

(51) Int. Cl. A01H 5/00; C12N 15/60; C07H 21/00; C08G 63/48; C07K 14/00; C12N 15/82; C12N 5/10

(52) U.S. Cl. 800/281; 536/23.2; 536/24.1; 435/320.1; 435/468; 435/419; 800/298; 800/306; 554/1; 530/350

(57) ABSTRACT

The present invention relates to the identification and characterization of a plant cyclopropane fatty acid synthase and the identification and cloning of the relevant gene sequence. The invention also relates to the use of that gene for the efficient production of cyclopropane fatty acids in an oilseed crop. The invention specifically relates to a cyclopropane fatty acid synthase from a plant in which the major cyclic fatty acids accumulated in the seed are cyclopropane fatty acids.

### FIELD OF INVENTION

The invention relates to the efficient production of cyclopropane fatty acids in plants. The production process particularly uses genetically modified plants.

### BACKGROUND

Plant oils have a wide range of compositions. The constituent fatty acids determine the chemical and physico-chemical properties of the oil which in turn determine the utility of the oil. Plant oils are used in food and increasingly in non-food industrial applications, particularly lubricants.

To reduce environmental impact, the production of efficient biodegradable lubricants has been contemplated. The starting materials for such lubricants are plant oils.

Classical plant oils from crops grown on a commercial scale typically contain saturated and unsaturated linear fatty acids with chain lengths between 12 and 18 carbon atoms. The physical properties of these fatty acids do not meet the requirements for high-performance lubricants.

To obtain a sufficient lubricant function, the carbon chains need to be long enough, probably around 16 to 18 carbon atoms. With saturated chains of this length the melting point and cloud point increase to unacceptable levels for use in car engines.

With the requirement for long chains, modifications of the saturated chain are required that reduce the melting point. In classical plant oils these modifications are desaturations, which lead to the desired properties as a lubricant. However, unsaturated fatty acids have an additional problem, in that they are oxidatively unstable, and therefore have a short functional life.

To address these problems, it has been shown that it is particularly advantageous to use branched chain fatty acids as a lubricant base (WO 99/18217). The synthetic route selected is the production of the intermediate cylopropane fatty acids in plant cells for conversion into branched chain fatty acids by industrial processing.

Cyclic fatty acids containing three carbon carbocyclic rings, especially cyclopropane fatty acids, are of particular industrial interest. The cyclopropane fatty acids have physical characteristics somewhere between saturated and monounsaturated fatty acids. The strained bond angles of the carbocyclic ring are responsible for their unique chemistry and physical properties. Hydrogenation allows the ring to open with the production of methyl-branched fatty acids. These branched fatty acids have the low temperature properties of unsaturated fatty acids and their esters without susceptibility to oxidation. Such branched fatty acids are therefore eminently suitable for use in lubricants.

Further they may be used as a replacement for “isostearate” a commodity in the oleochemical industry which is included in the formulation of cosmetics and lubricant additives, for example. The highly reactive nature of the strained ring also encourages a diverse range of chemical interactions allowing the production of numerous novel oleochemical derivatives.

It has previously been demonstrated that it was possible to introduce a cyclic fatty acid synthase (CFAS) gene from E. coli into tobacco cells and in this way produce cyclic fatty acids in plant cells (WO 99/18217 and U.S. Pat. No. 5,936,139). However, the amount of CFA produced was quite low and this is not a commercially viable production route.

Although the biosynthesis of CFA in bacteria is well understood, their synthesis in plants remains largely unknown.

Cyclic fatty acids (especially cyclopropane fatty acids) are rather unusual in plants. Although as early as 1978 and 1980, respectively, cyclopropenes and cyclopropanes had been identified in few plant seeds, their biochemical synthesis has not been elucidated.

Schmid (U.S. Pat. No. 5,936,139) acknowledges that cyclic fatty acids are a significant component of Lychee and Sterculia oils; using them as qualitative standards when analyzing oil extracted from tobacco tissue transformed with the E. coli CFAS. U.S. Pat. No. 5,936,139 recommends the expression of a microbial gene in an oilseed crop because the bacterial pathway is understood and would thus not suggest to one skilled in the art to use a CFAS gene from a plant source as the plant synthetic pathway is unknown.

Allen et al (WO 99/43827) were able to identify maize, rice, wheat, soya and bean EST sequences by homology to microbial sequences. They were not able to demonstrate any biochemical activity or relevant fatty acid content in transgenic plant tissue.

Most recently a CFAS has been identified and characterized in Sterculia foetida (WO 03/060079).

Sterculia bears small oil-rich seeds (55% by dry weight) commonly known as Java olives that are consumed especially in the Far East. The seeds are very rich in cyclopropene fatty acids (up to 78% of fatty acids), especially sterculate, some 65% or more.

Bao et al (WO 03/060079) have successfully isolated and cloned the gene coding for the CFAS and expressed it in undifferentiated tobacco tissue. Interestingly the Sterculia CFAS has two enzymatic domains and it is postulated that whilst the carboxy terminal contains the CFAS domain and catalyses the synthesis of dihydrosterculate, the amino terminus contains an oxidase which is capable of completing the synthesis of sterculate by a desatuartion reaction. When expressed in tobacco tissue a significant but still low level of dihydrosterculate (mean of 4%) was detected.

This incomplete reaction may suggest that the isolated gene is not fully functional. Indeed, and as suggested by Bao et al (Proc; Natl Acad, Sci USA, 2002, 99(10), 7172-7) the CFAS gene of Sterculia would have been expected to be fully functional as Sterculia produces a very large amount of cyclopropene fatty acids, and these are products of desaturation of cyclopropane fatty acids (see also Yano et al, Lipids, 1972, 7; 35-45). Thus, the quantity of the intermediate product was expected to be high in the absence of degrading enzymes.

It remains therefore difficult to predict whether it is possible to identify a CFAS gene from a plant source which, when introduced into an organism, and in particular an oil crop plant, would code for an enzyme interacting more efficiently with the cellular machinery and available substrates to produce CFA in sufficiently high quantities.

As the mechanism of CFA synthesis in plants can only be speculated, it remains difficult to anticipate the efficiency of production of cyclopropanes in plant seeds.

As indicated by Bao and Schmidt (op. cit.) Lychee is one plant that is known to have a high percentage of cyclopropane fatty acid in its seeds (over 40% cyclopropane fatty acid, specifically dihydrosterculate) although this is a non-oily seed, (oil being perhaps only 1% dry weight).

Although the final products produced by Sterculia and Lychee are different (cyclopropenes vs cyclopropanes respectively), one could believe that the biochemical pathway for production of dihydrosterculate could be similar, the main difference being the demonstrated presence of desaturating enzymes in Sterculia, and a possible absence of such enzymes in Lychee.

In fact, in the view of the data obtained by Bao, who indicates that the CFAS from Sterculia is not efficient as it was thought to be, one could be also skeptical about the chances of obtaining efficiency of a CFAS coming from Lychee and thus a good cyclopropane fatty acid production. Furthermore, this enzyme was not characterized, and thus one would not have been enticed to look for it.

The inventors have now identified in Lychee a nucleic acid sequence that codes for a protein that has CFA synthase activity. Surprisingly, this protein is part of a larger protein and this part on its own demonstrates a very powerful ability to produce cyclopropane fatty acids. This nucleic acid sequence can thus be very useful for the efficient production of cyclopropane fatty acids in plants, in particular the seeds, of especially high oil-producing crop plants.

### SUMMARY OF THE INVENTION

The present invention relates to the identification and characterization of a plant cyclopropane fatty acid synthase and the identification and cloning of the relevant gene sequence. The invention also relates to the use of that gene for the efficient production of cyclopropane fatty acids in an oilseed crop.

The invention specifically relates to a cyclopropane fatty acid synthase from a plant in which the major cyclic fatty acids accumulated in the seed are cyclopropane fatty acids.

### FIGURES

FIG. 1: Nucleotide sequence of the LsCFAS1 gene (SEQ ID N^{o} 3). The first and last codons of the translated region are underlined.

FIG. 2: Amino acid sequence of LsCFAS1 (SEQ ID N^{o} 4).

FIG. 3: Nucleotide sequence of the LsCFAS2 gene (SEQ ID N^{o} 1). The leucine codon, artificially converted to a methionine codon, to become the translational start of the LsCFAS2 carboxy domain construct is indicated in bold. The first and last codons of the translated region are underlined.

FIG. 4: Amino acid sequence of LsCFAS2 (SEQ ID N^{o} 2). The leucine residue, artificially converted to methionine, to become the start of the LsCFAS2 carboxy domain protein is indicated in bold.

FIG. 5: Amino acid sequence of LsCFAS2 carboxy domain (SEQ ID N^{o} 5).

FIG. 6: RT-PCR of LsCFAS2 carboxy domain in E. coli. Lane 1; positive control (plasmid DNA), lanes 2 and 3; LsCFAS2 carboxy domain, 90 min and 4 hr IPTG induction respectively, lanes 4 and 5; E. coli CFAS, 90 min and 4 hr IPTG induction respectively.

FIG. 7: Gas Chromatograph of lipids extracted from E. coli expressing LsCFAS2 carboxy domain

FIG. 8: Gas Chromatograph of lipids extracted from E. coli expressing full-length LsCFAS2.

### DESCRIPTION

One aspect of the invention relates to an isolated nucleic acid encoding a cyclopropane fatty acid synthase isolated from a plant in which the major (cyclic) fatty acids accumulated in the seeds are cyclopropane fatty acids.

In particular, said plant is from the family of Sapindaceae.

The Sapindaceae are members of an interesting family mainly found in the tropics. The only two plants identified to date that have seeds in which cyclopropane fatty acids accumulate without any cyclopropene fatty acids belong to this family. Litchi sinensis (Lychee) and Euphoria longana (Longan) are both eaten as tropical fruits and do not have seeds with a high oil content.

In the preferred embodiment, said isolated nucleic acid codes for a protein having at least 80%, more preferably 90%, more preferably 95% identity with SEQ ID N^{o} 2 (Lychee LsCFAS2 protein), which harbors CFA synthase activity, when introduced into E. coli or in a plant, especially oilseed rape or linseed.

As indicated in the examples, Lychee contains two proteins that show homology with CFAS from other plants and bacteria. The inventors have demonstrated that only one of these two proteins is able to generate CFA (cyclopropanes) in relatively high amounts. This results from the expertise of the inventors in performing the search for the CFA synthase in Lychee.

As a preferred embodiment, the invention relates to an isolated nucleic acid that encodes a protein that is at least 80% identical to SEQ ID N^{o} 2.

Two polynucleotides or polypeptides are said to be “identical” if the sequence of nucleotides or amino acid residues, respectively, in the two sequences is the same when aligned for maximum correspondence as described below.

Sequence comparisons between two (or more) polynucleotides or polypeptides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2: 482 (1981), by the homology alignment algorithm of Neddleman and Wunsch, J. Mol. Biol. 48: 443 (1970), by the search for similarity method of Pearson and Lipman, Proc. Natl. Acad. Sci. (U.S.A.) 85: 2444 (1988), by computerized implementation of these algorithms (GAP, BESTFIT, BLAST N, BLAST P, FASTA, and TFASTA in the Wisconsin Genetics Software Package, Genetics Computer Group (GCG), 575 Science Dr., Madison, Wis.), or by inspection.

Preferably, the percentage of identity of two polypeptides is obtained by performing a blastp analysis with the sequence encoded by the nucleic acid according to the invention, and SEQ ID N^{o} 2, using the BLOSUM62 matrix, with gap costs of 11 (existence) and 1 (extension).

The percentage of identity of two nucleic acids is obtained using the blastn software, with the default parameters as found on the NCBI web site (http://www.ncbi.nlm.nih.gov/BLAST/).

“Percentage of sequence identity” is also determined by comparing two optimally aligned sequences over a comparison window, where the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

In another embodiment, the invention relates to an isolated nucleic acid comprising a sequence that is greater than 80%, preferably greater that 90%, more preferably greater than 95%, more preferably greater than 97, 98 or 99% identical to any of:
SEQ ID NO 1 (Lychee LsCFAS2, nucleic),
nucleotides 37-2655 of SEQ ID N^{o} 1,
a sequence from between nucleotides 1197 and 1371 to nucleotide 2655 of SEQ ID N^{o} 1,
the sequence from nucleotide 1282 to 2655 of SEQ ID N^{o} 1,

and that codes for an active CFA synthase.

In a preferred embodiment, said isolated nucleic acid comes from Litchi sinensis or a plant of the family of Sapindaceae.

More preferably said nucleic acid comprises nucleotides 37-2655 of SEQ ID N^{o} 1, or comprises the sequence starting from between nucleotides 1197 and 1371 and finishing at nucleotide 2655 of SEQ ID N^{o} 1. In particular, is encompassed by the invention, a nucleotide sequence that is a fragment of SEQ ID N^{o} 1, that comprises nucleotides 1282-2655 of SEQ ID N^{o} 1, and that codes for a CFAS.

Another aspect of the invention relates to an isolated nucleic acid sequence encoding the amino acid sequence of the carboxy terminus of a cyclopropane fatty acid synthase isolated from a plant in which the major fatty acids accumulated in the seeds are cyclopropane fatty acids.

The inventors have indeed demonstrated that, in these plants, only part of a broader sequence can have CFAS activity by itself.

The inventors were able to correctly identify the functional delineation between two domains within these proteins, and demonstrated that it was possible to express one of the domains without loss of CFAS activity of the expressed protein. Thus, surprisingly, the inventors were able to identify and clone an active CFAS domain, which protein was stable, folded correctly, associated with necessary cofactors and therefore functioned in the anticipated and desired manner.

As exemplified, two CFAS genes have been identified. Both have similar homology to the well characterized E. coli CFAS gene and the CFAS domain of the Sterculia foetida gene. One gene (LsCFAS1) encodes a protein of a similar size to the E. coli CFAS, 356 amino acid residues, but no CFAS activity was associated with this protein. The second gene (LsCFAS2) encodes a larger protein, 870 amino acid residues. The lack of activity associated with LsCFAS1 suggested that the extra 5′ region of CFAS2 was essential for CFAS activity. Surprisingly the LsCFAS2 3′ region, encoding a protein of similar size to the E. coli CFAS and LsCFAS1, was, by itself, associated with CFAS activity in the absence of the aforementioned extra 5′ region.

Thus, a particular embodiment of the invention relates to an isolated nucleic acid comprising a sequence encoding a fragment of the amino acid sequence set forth in SEQ ID NO: 2, wherein said fragment has CFAS activity.

A preferred embodiment encompasses an isolated nucleic acid comprising the sequence encoding between 400 and 458 of the last amino acids of the sequence set forth in SEQ ID NO: 2.

An isolated nucleic acid comprising the sequence encoding the last 458 amino acids of the sequence set forth in SEQ ID NO: 2 is a most preferred embodiment

Another aspect of the invention relates to a chimeric gene comprising a nucleic acid sequence according to the invention operatively linked to suitable regulatory sequences for functional expression in plants, and in particular in the seeds of oil plants. The phrase “operatively linked” means that the specified elements of the component chimeric gene are linked to one another in such a way that they function as a unit to allow expression of the coding sequence. By way of example, a promoter is said to be linked to a coding sequence in an operational fashion if it is capable of promoting the expression of said coding sequence. A chimeric gene according to the invention can be assembled from the various components using techniques which are familiar to those skilled in the art, notably methods such as those described in Sambrook et al. (1989, Molecular Cloning, A Laboratory Manual, Nolan C., ed., New York: Cold Spring Harbor Laboratory Press). Exactly which regulatory elements are to be included in the chimeric gene will depend on the plant and the type of plastid in which they are to work: those skilled in the art are able to select which regulatory elements are going to work in a given plant.

In order to produce a significant quantity of cyclic fatty acids in plant tissues it is much preferable to drive the expression of the newly identified CFAS gene with a suitable plant promoter. Many promoters are known and include constitutive and tissue and temporally specific.

For expressing the protein in another organism, such as a microorganism or another eukaryotic cell, suitable promoters are well known in the art.

Promoter sequences of genes which are expressed naturally in plants can be of plant, bacterial or viral origin. Suitable constitutive promoters include but are not restricted to octopine synthase (Ellis et al, 1987, EMBO J. 6, 11-16; EMBO J. 6, 3203-3208), nopaline synthase (Bevan et al, Nucleic Acids Res. 1983 Jan. 25; 11(2):369-85), mannopine synthase (Langridge et al, PNAS, 1989, vol. 86, 9, 3219-3223) derived from the T-DNA of Agrobacterium tumefaciens; CaMV35S (Odell et al, Nature. 1985 Feb 28-Mar 6; 313(6005):810-2) and CaMV19S (Lawton et al Plant Mol. Biol. 9:315-324, 1987) from Cauliflower Mosaic Virus; rice actin (McElroy et al, Plant Cell, 2:163-171, 1990), maize ubiquitin (Christensen et al, 1992, Plant Mol Biol 18: 675-689) and histone promoters (Brignon et al, Plant J. 1993 September; 4(3):445-57) from plant species. Sunflower ubiquitin promoter is also a suitable constitutive promoter, Binet et al., 1991, Plant Science, 79, pp 87-94).

It is preferable that the CFAS gene is expressed at a high level in an oil producing tissue to avoid any adverse effects of expression in plant tissues not involved in oil biosynthesis and also to avoid the waste of plant resources; commonly the major oil producing organ is the seed.

Thus, in a preferred embodiment, the chimeric gene of the invention comprises a seed specific promoter operatively linked to the nucleic acid of the invention. Suitable promoters include but are not limited to the most well characterised phaseolin (Sengupta-Gopalan et al., 1985, Proc Natl Acad Sci USA 85: 3320-3324), conglycinin (Beachy et al., 1985, EMBO J. 4: 3407-3053), conlinin (Truksa et al, 2003, Plant Phys and Biochem 41: 141-147), oleosin (Plant et al., 1994, Plant Mol Biol 25(2): 193-205), and helianthinin (Nunberg et al., 1984, Plant Cell 6: 473-486).

In a very preferred embodiment, said promoter is the Brassica napus napin promoter (European patent No 0255278), being seed specific and having an expression profile compatible with oil synthesis.

In another very preferred embodiment, said promoter is from a FAE (Fatty acid Elongase; WO2/052024).

The invention also relates to a transformation vector, in particular a plant transformation vector comprising a nucleic acid molecule or a chimeric gene according to the invention. For direct gene transfer techniques, where the nucleic acid sequence or chimeric gene is introduced directly into a plant cell, a simple bacterial cloning vector such as pUC19 is suitable. Alternatively more complex vectors may be used in conjunction with Agrobacterium-mediated processes. Suitable vectors are derived from Agrobacterium tumefaciens or rhizogenes plasmids or incorporate essential elements from such plasmids. Agrobacterium vectors may be of co-integrate (EP-B-0 116 718) or binary type (EP-B-0 120 516).

The invention also relates to a method for expressing a plant cyclopropane fatty acid synthase in a host cell, in particular a plant cell comprising transforming said cell with an appropriate transformation vector according to the invention. In the case of a plant cell, one would be transfecting a suitable plant tissue with a plant transformation vector. Integration of a nucleic acid or chimeric gene within a plant cell is performed using methods known to those skilled in the art. Routine transformation methods include Agrobacterium-mediated procedures (Horsch et al, 1985, Science 227:1229-1231). Alternative gene transfer and transformation methods include protoplast transformation through calcium, polyethylene glycol or electroporation mediated uptake of naked DNA. Additional methods include introduction of DNA into intact cells or regenerable tissues by microinjection, silicon carbide fibres or most widely, microprojectile bombardment. All these methods are now well known in the art.

A whole plant can be regenerated from a plant cell. A further aspect relates to a method for expressing a plant cyclopropane fatty acid synthase in a plant comprising transfecting a suitable plant tissue with a plant transformation vector and regeneration of an intact fully fertile plant. Methods that combine transfection and regeneration of stably transformed plants are well known.

Thus a further aspect of the invention relates to a plant transformed with a heterologous cyclopropane fatty acid synthase. Any plant that can be transformed and regenerated can be included. An embodiment relates to a plant where the original plant is an oil producing crop plant. Preferred plants include the oilseed crops such as rape, linseed, sunflower, safflower, soybean, corn, olive, sesame and peanuts. Most preferred are plants that produce oleic acid.

Transformation methods are known for sunflower such as those described in WO 95/06741 and more recently Sankara Rao and Rohini, (1999, Annals of Botany 83: 347-354). Linseed transformation was first achieved in 1988 by Jordan and McHughen (Plant cell reports 7: 281-284) and more recently improved by Mlynarova et al (Plant Cell reports, 1994, 13: 282-285)

A most preferred embodiment is a plant transformed with a heterologous cyclopropane fatty acid synthase where the original plant is Brassica napus. This can be achieved by known methods such as Moloney et al, Plant cell reports 8: 238-242, 1989.

Another aspect of the invention relates to the oil produced by a plant transformed with a heterologous cyclopropane fatty acid synthase. A preferred embodiment is an oil having an increased proportion of cyclopropane fatty acids. A most preferred embodiment is an oil having an increased proportion of dihydrosterculic acid.

### EXAMPLES

All DNA modifications and digestions were performed using enzymes according to the manufacturers' instructions and following protocols described in Sambrook and Russell, 2001; Molecular Cloning, A Laboratory Manual.

### Example 1

### Identification and Cloning of Lychee CFAS Genes

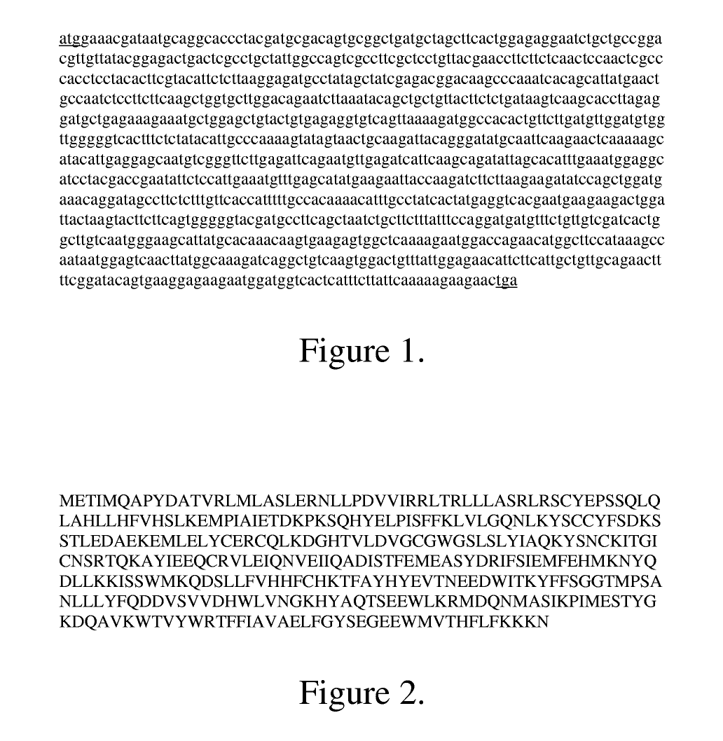

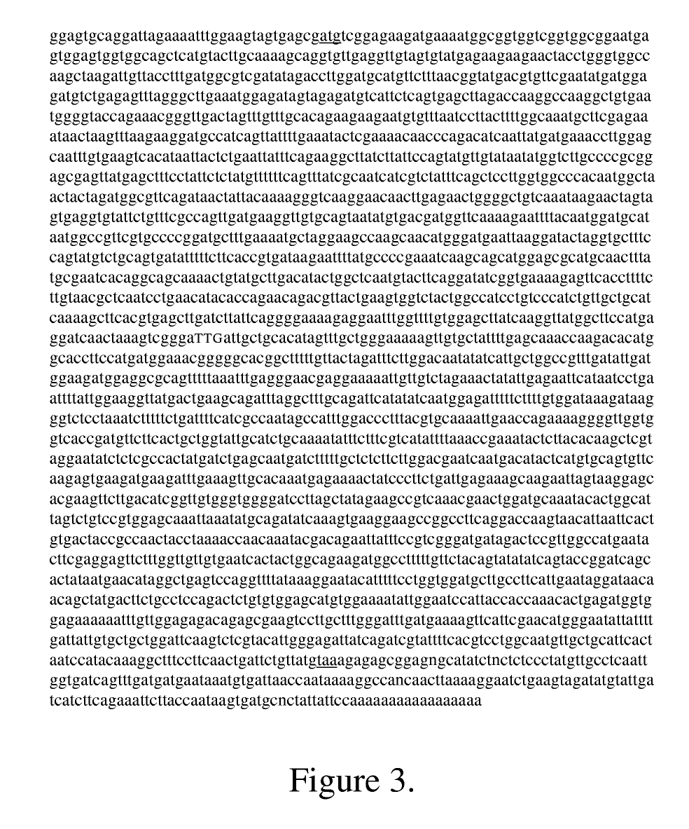

The inventors have identified two putative CFAS genes expressed in Lychee immature seed; LsCFAS1 (FIG. 1, SEQ ID N^{o} 3) and LsCFAS2 (FIG. 3, SEQ ID N^{o} 1).

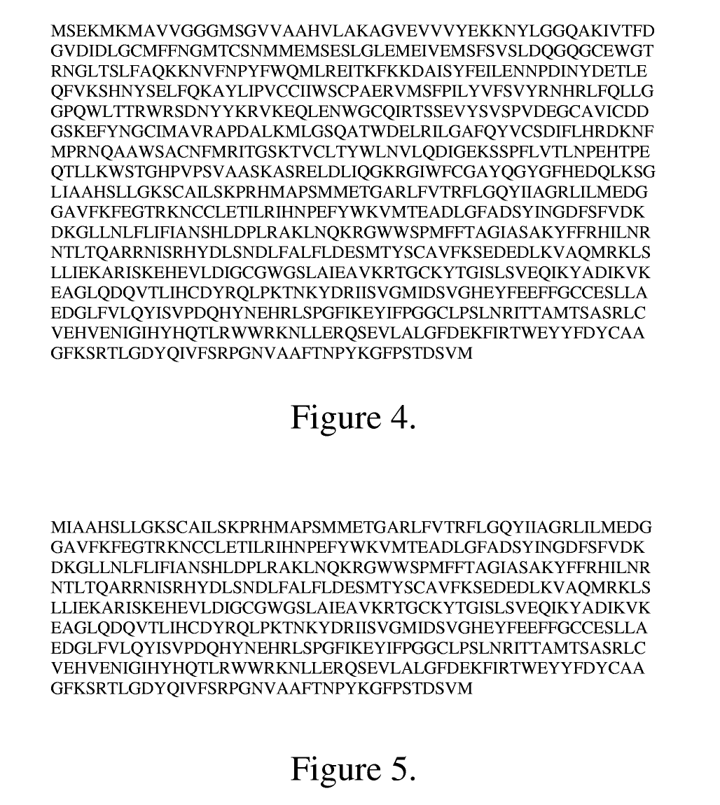

LsCFAS1 encodes a protein of 356 amino acid residues (FIG. 2, SEQ ID N^{o} 4) and has 38% homology with E. coli CFAS
LsCFAS2 encodes a protein of 870 amino acid residues (FIG. 4, SEQ ID N^{o} 2) and has 47% homology to E. coli CFAS.

### Example 2

### Functional validation of LsCFAS1 in E. coli

A full length clone of LsCFAS1, pEW50, in a basic cloning vector was prepared. In order to facilitate detection of expression of this gene in E. coli, an N-terminal His tag was added to the synthesized protein by introducing the coding region into a suitable expression vector (pQE81). Protein produced in this way could be analysed for its ability to synthesise cyclic fatty acids.

### i) Transformation

E .coli (DH5α, BL21 Gold, mutant strain YY1273 described by Chang and Cronan, 1999) was transformed with the above plasmid. Transformants were grown in LB medium containing 150 μg mL⁻¹ carbenicillin at 37° C. Expression of CFAS gene was induced at midlog phase by adding IPTG to a final concentration of 1 mM and incubating for 2 hours at 28° C. The cells were harvested by centrifugation and the pellet was used for purification of CFA synthase.

### ii) Extraction And Purification of Protein

The induced cells were harvested by centrifugation (6 000 g, 15 min, 4° C.). The cells were incubated in lysate buffer (Quiagen: Phosphate buffer, pH 8 containing NaCl-200 mM and imidazol 20 mM) and then ground in the same buffer and in liquid nitrogen. After centrifugation at 10 000 g, 20 min at 4° C., the CFA synthase was purified on Ni-NTA resin following the protocol recommended by Quiagen. The CFA synthase was concentrated X6 on microcentrifuged filters NMWL 5 000 (Sigma). The protein was detected by Western blotting. Sufficient protein was synthesised to carry out an assay for CFAS activity. No activity was detected.

iii) Fatty Acid and Lipid Analysis

Bligh and Dryer's method (1959) was used to extract lipids of bacterial cultures. 100 μL of bacterial culture were mixed with 375 μL of CHCl₃/Methanol (1:2, vol/vol). 100 μL of CHCl₃ was then added and mixed on rotary mixer. 100 μL of water was added and rapidly mixed. The mixture was then centrifuged for 30 s at 3000 rpm and the bottom phase was collected. The fatty acids were then esterified by TMAH using the following described protocol. 100 μL of ether were added to the lipid extract and mixed. 10 μL of TMAH were added and incubated 5 min at room temperature. The mixture was centrifuged for 30 s at 3000 rpm for phase separation and the upper phase was collected and concentrated under a nitrogen flux. A sample was analyzed by GC. No cyclic fatty acids were detected.

### Example 3

### Functional Validation of LsCFAS1 in Brassica napus

The coding region from the full length clone of LsCFAS1, pEW50, was used to create pEW51, a basic cloning vector carrying an expression cassette driven by the napin promoter. The expression cassette was transferred to a suitable binary vector SCVnosnptII to create pEW52, which in turn was introduced into the A.tumefaciens strain C58 pMP90.

Transgenic rape plants were produced with the A.tumefaciens carrying pEW52 according to the method of Moloney et al, 1989. Expression of the transgene was confirmed by RT-PCR.

Lipids were extracted from immature seed collected from 11 individual transgenic rape plants and the fatty acid profile determined by GC. No cyclic fatty acids were detected.

In conclusion, a Lychee cDNA clone was readily identified with significant homology to microbial CFAS. A full length clone was expressed in B.napus under the control of a suitable strong seed-specific promoter. Good expression was confirmed by RT-PCR but analysis of oil extracted from transgenic rape seed failed to detect any cyclic fatty acids.

### Example 4

### Functional Validation of LsCFAS2 Carboxy Domain in E. coli

LsCFAS2 was initially represented by several partial cDNA clones due to its double domain and hence great length. The CFAS domain is positioned towards the carboxy terminus of the protein (FIG. 5) and hence the 3′ portion of the coding region. A partial clone of LsCFAS2 cDNA, in a basic cloning vector, was identified having a complete CFAS coding domain. In order to facilitate detection of expression of this domain in E. coli, an N-terminal His tag was added to the synthesized protein by introducing the CFAS coding domain into a suitable expression vector (pQE81) to create pEW56B.

Bacterial transformation, protein extraction and purification and CFAS activity and lipid analysis were carried out as in Example 2.

Due to the problems initially encountered with the expression of LsCFAS1 in E. coli and detection of significant protein, RT-PCR was carried out to confirm that expression was detectable at the messenger RNA level. Bacteria were grown overnight in 100 ml of prewarmed LB medium containing 100 μg/ml carbenicillin at 37° C. with shaking at 210 rpm, until the OD₆₀₀ was 0.5-0.7. Expression was induced by adding IPTG to final concentration of 1 mM. After further growth for 90 min or 4 hr 3 ml samples were collected, centrifuged at 10 000 g for 10 min at 4° C. and frozen. RNA was extracted by thawing the cell pellet for 15 min on ice and resuspending in 100 μl of lysozyme-TE buffer. After incubation at room temperature for 10 min, the RNA was purified using an RNeasy Mini Kit (Qiagen). RT-PCR is performed using Titan one tube RT-PCR Kit (ROCHE).

| Cycles | Temperature and time |
|---|---|
| 1X | 50° C. for 30 min |
| 1X | 94° C. for 2 min |
| 10X | 94° C. for 30 s |
| | 54° C. (LsCFAS carboxy domain) |
| | or 62° C. (E. coli CFAS); for 30 s |
| | 68° C. for 1 min |
| 25X | 94° C. for 30 s |
| | 54° C. (LsCFAS carboxy domain) |
| | or 62° C. (E. coli CFAS); for 30 s |
| | 68° C. for 1 min, cycle elongation of 5 s for each cycle |
| | (e.g., cycle n °11 has additional 5 s, cycle n °12 has |
| | additional 10 s . . . ) |
| 1X | 68° C. for 10 min |

The PCR products were separated on a 1% agarose gel.

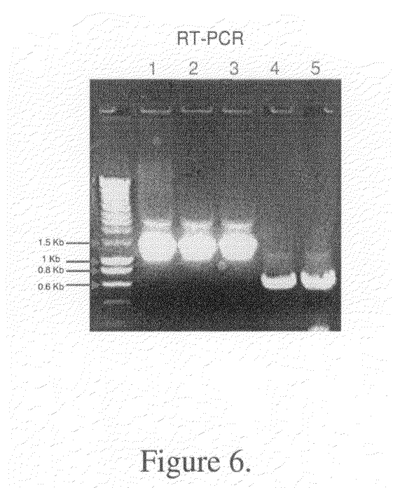

RT-PCR provided evidence of strong expression in E. coli (FIG. 6)

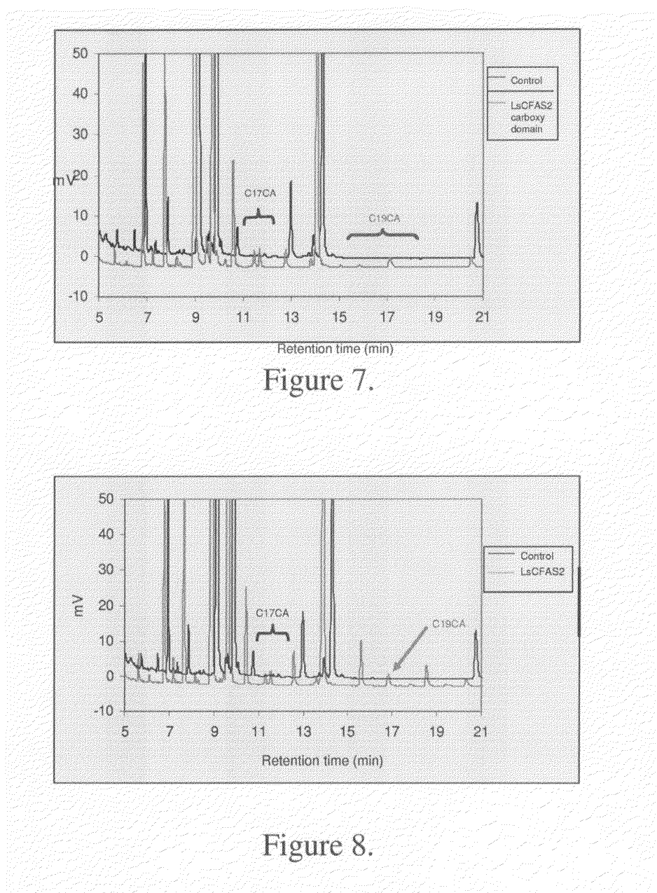

Extracted lipids were analysed by GC on a polar column (BP*70 60 m) and significant amounts of C17CA were detected along with trace amounts of C19CA (FIG. 7).

### Example 5

### Functional Validation of LsCFAS2 Carboxy Domain in Tobacco Suspension

The clone of the CFAS coding domain pEW56B described above was used as the starting point to create a suitable construct for expression in tobacco. The coding region was used to create pEW51 an expression cassette driven by the constitutive CaMV 35S promoter. The expression cassette was transferred to a suitable binary vector, which in turn was introduced into the A. tumefaciens strain.

### i) Culture and Transformation of Tobacco

Tobacco suspension cells (Nicotiana tabacum L. cv Bright Yellow-2: BY2) were cultivated in liquid LS medium at 25° C. and in dark conditions (Linsmaïer and Skoog, 1965). Cultures were subcultured weekly with 5% (vol/vol) inoculum from a 7-day-old culture and shaken in 250 mL flasks (110 rpm).

### Transformation Protocol:

10 mL of a tobacco BY2 suspension cells (3-day-old culture) was infected with 500 μL of recombinant Agrobacterium tumefaciens. The cocultivation was maintained 2 days in LS medium at 25° C. without shaking. The cells were collected after centrifugation at 50 g during 3 min. The excess bacteria were removed by washing the BY2 cells in LS medium 2-3 times. The plant cells were then plated on solid LS medium complemented with kanamycin (100 μg/μL) and cefotaxime (250 μg/μL). Transgenic calli were subcultured every 3 weeks on fresh solid medium containing kanamycin and cefotaxime.

### ii) Extraction and Purification of the Protein:

The cells were suspended in Hepes 80 mM pH 6.8 with saccharose 0.33 M, containing EDTA 1 mM, β-mercaptoethanol 10 mM and PVP 1%. The cells were disrupted by grinding in liquid nitrogen. The resulting lysate was centrifuged at 10 000 g for 20 min at 4° C. and the supernatant was used for activity assays. The protein content was determined by the Bradford method (Bradford, 1976).

All subsequent purification steps were performed at 0-4° C.

iii) Fatty Acid and Lipid Analysis:

1 g of BY2 cells were dried at 50° C. overnight and then ground to a fine powder.

2 mL of trimethylpentane were added to the powder, the mixture was centrifuged at 13 000 g during 30 s and the supernatant was dried under a nitrogen flux. 100 μL of ethylether and 5 μL of TMAH (tetramethyl ammonium 20% in methanol) were added to 2 mg of oil and mixed on rotary mixer. 50 μL of trimethylpentane were added to the previous mixture and mixed. The mixture was centrifuged at 13 000 g during 30 s and the supernatant was dried and the extract was dissolved in 2 to 5 μL of trimethylpentane.

Preliminary analysis by GC-MS of a selection of 12 transformed tobacco calli, confirmed by PCR and RT-PCR, revealed a fatty acid profile significantly different from that of control tobacco cells. Trace amounts of cyclic fatty acids were detected (Table 1).

TABLE 1
GC-MS analysis of tobacco cells transformed with LsCFAS2
carboxy domain. % cyclic FAMEs content/total FAMEs.

| Sample | % C17CA | % C19CA |
|---|---|---|
| Control | 0.038 | n.d. |
| 1 | 0.133 | 0.117 |
| 3 | 0.075 | 0.082 |
| 5 | 0.09 | 0.049 |
| 6 | 0.111 | 0.042 |
| 9 | 0.094 | n.d. |
| 2 | 0.049 | 0.199 |
| 11 | n.d. | n.d. |
| 7 | 0.051 | 0.045 |
| 3 | 0.072 | n.d. |
| 8 | 0.111 | n.d. |
| 4 | 0.041 | 0.227 |
| 12 | n.d. | 0.02 |

n.d: not detected

### Example 6

### Functional Validation of LsCFAS2 Carboxy Domain in Brassica napus

The clone of the CFAS coding domain pEW56B described above was used as the starting point to create a suitable construct for expression in oilseed rape. The coding domain was subcloned into a Gateway Entr vector to create pEW79 which was subsequently recombined into the Gateway destination vector, thus creating pEW80-SCV. In this one step an expression cassette driven by the napin promoter is created in a binary vector suitable for oilseed rape transformation. Plasmid pEW80-SCV was introduced into the A. tumefaciens strain C58 pMP90

Transgenic rape plants are produced with the A. tumefaciens carrying pEW80-SCV according to the method of Moloney et al, 1989. Expression of the transgene is confirmed by RT-PCR.

RNA is isolated from ten 30 day seeds using the RNeasy kit (Qiagen) with on-column DNase digestion following the protocol from the manufacturer.

Two Lcfa2′ primers, P18-P4 or LcfaTrev, are annealed to samples of 1 ug RNA, in addition to an endogenous control primer, RESrev, targeted against the B.napus acyltransferase-1 gene. 0.5 ug of each specific primer is used per reaction. Reverse transcriptase reactions are then carried out in a volume of 25 ul using ImPromII RT or MMLV RT (both Promega) with the buffers supplied, for 1 hr at 42° C. An aliquot of 5 ul is then used as a template in the PCR reaction using Taq polymerase (Bioline) with an annealing temperature of 60° C. and 3 mM MgCl₂. The same reverse primers are again used in the PCR reaction together with forward primers P18-P1 or RESfor. Products are analysed by agarose gel electrophoresis and the relative expression level assessed visually.

### Primer Sequences:

| | |
|---|---|
| Reverse primer P18-P4: | |
| AAACTGCGCCTCCATCTTCCATC | (SEQ ID N^{o} 6) |
| Fwd primer P18-P1: | |
| TCATGATTGCTGCACATAGTTTGCTGG | (SEQ ID N^{o} 7) |
| RT-PCR product size: | 171 bp |
| Reverse primer LcfaTrev: | |
| AGATGCAATACCAGCAGTGAAG | (SEQ ID N^{o} 8) |
| Forward primer P18-P1: | |
| TCATGATTGCTGCACATAGTTTGCTGG | |
| RT-PCR product size: | 440 bp |
| Reverse primer RESrev: | |
| CGAGTGACACTTGATGTGAACATGC | (SEQ ID N^{o} 9) |
| Forward primer RESfor: | |
| GGTCAGGTTGCCTAGGAAGC | (SEQ ID N^{o} 10) |
| RT-PCR product size: | 424 bp |

Lipids are extracted from immature seed collected from individual transgenic rape plants and the fatty acid profile determined by GC.

### Example 7

### Functional Validation of Full Length LsCFAS2 in E. coli

The complete LsCFAS2 sequence was initially cloned as three overlapping fragments. These fragments were used to create a full length clone, pEW86, in a basic cloning vector. The coding region was introduced into a suitable expression vector (pBAD). Protein produced in this way could be analysed for its ability to synthesise cyclic fatty acids.

Bacterial transformation, protein extraction and purification and CFAS activity and lipid analysis were carried out as in Example 2.

Extracted lipids were analysed by GC on a polar column (BP*70 60 m) and significant amounts of C17CA were detected along with trace amounts of C19CA (FIG. 8).

1. An isolated nucleic acid encoding a cyclopropane fatty acid synthase isolated from a plant, wherein said cyclopropane fatty acid synthase comprises:
a. the sequence encoding between 400 and 458 of the last amino acids of the amino acid sequence set forth in SEQ ID NO: 2.
b. a sequence that is at least 80%, 85%, 90%, 95%, 97%, 98%, 99%, identical to the sequence in a., wherein said sequence codes for a protein having cyclopropane fatty acid synthase activity
c. a fragment of the sequence in a. or b., wherein said fragment codes for a protein having cyclopropane fatty acid synthase activity.

2. The isolated nucleic acid of claim 1 where the plant is Litchi sinensis.

3. The nucleic acid of claim 1, comprising a sequence that is greater than 80%, identical to any of:
a. SEQ ID NO 1 (Lychee LsCFAS2, nucleic),
b. nucleotides 37-2655 of SEQ ID N^{o} 1,
c. a sequence from between nucleotides 1197 and 1371 to nucleotide 2655 of SEQ ID N^{o} 1,
d. the sequence from nucleotide 1282 to 2655 of SEQ ID N^{o} 1,

4. The isolated nucleic acid of claim 1, encoding a cyclopropane fatty acid synthase comprising the sequence encoding the amino acid sequence set forth in SEQ ID NO 2, or a sequence that is at least 80% identical to SEQ ID NO 2.

5. A chimeric gene comprising a nucleic acid sequence of claim 1, linked to suitable regulatory sequences for functional expression.

6. The chimeric gene of claim 5 wherein said regulatory sequence comprises a seed specific promoter.

7. The chimeric gene of claim 6, comprising the Brassica napus napin promoter.

8. A plant transformation vector comprising a nucleic acid sequence of claim 1.

9. A plant transformation vector comprising a chimeric gene of claim 5.

10. A method for expressing a plant cyclopropane fatty acid synthase in a plant cell comprising
a. providing a vector of claim 8
b. transfecting said plant cell with said vector

11. A plant cell transformed with a vector according to claim 8.

12. A method for producing a fertile plant expressing a plant cyclopropane fatty acid synthase comprising the steps of
a. providing a vector according to claim 8
b. transfecting a suitable plant tissue with the vector
c. regenerating a fertile plant expressing a plant cyclopropane fatty acid synthase.

13. A plant comprising a cell transformed with a vector according to claim 8.

14. The plant of claim 13 where the original plant is an oil producing crop plant.

15. The plant of claim 14 being from the Brassica napus species.

16. Oil from the transgenic plant of claim 13

17. The oil of claim 16 having an increased proportion of cyclopropane fatty acids as compared to oil isolated from a non-transformed plant.

18. A cyclopropane fatty acid synthase polypeptide from a plant in which the major fatty acids accumulated in the seeds are cyclopropane fatty acids.

19. A protein having cyclopropane fatty acid synthase activity isolated from a plant, comprising:
a. between 400 and 458 of the last amino acids of the amino acid sequence set forth in SEQ ID NO: 2.
b. a protein that is at least 80%, 85%, 90%, 95%, 97%, 98%, 99%, identical to the sequence in a.,
c. a fragment of the sequence in a. or b.

20. The isolated protein of claim 19 where the plant is Litchi sinensis.

21. The isolated protein of claim 19, having cyclopropane fatty acid synthase activity comprising the amino acid sequence set forth in SEQ ID NO 2, or a sequence that is at least 80% identical to SEQ ID NO 2.

21. (canceled)

22. A method for increasing the production of cyclopropane fatty acid in an organism comprising the step of transforming the organism with a vector comprising a nucleic acid sequence of claim 8.
